(19) United States
(12) Patent Application Publication
(10) Pub. No.: US 2015/0122734 A1
(43) Pub. Date: May 7, 2015

(54) Light Activated Cation Separation

(71) Applicant: Rutgers, The State University of New Jersey, New Brunswick, NY (US)

(72) Inventors: Richard E. Riman, Belle Mead, NJ (US); Brook Porter, Menlo Park, CA (US)

(73) Assignee: RUTGERS, THE STATE UNIVERSITY OF NEW JERSEY, New Brunswick, NJ (US)

(21) Appl. No.: 14/406,261

(22) PCT Filed: Jun. 7, 2013
(86) PCT No.: PCT/US13/44662

§ 371 Date: Dec. 8, 2014

Related U.S. Application Data
(60) Provisional application No. 61/657,341, filed on Jun. 8, 2012.

(51) Int. Cl. C22B 3/26; C22B 3/42; C02F 1/32; C07D 323/00; C02F 1/42

(52) U.S. Cl. CPC C22B 3/0005; C07D 323/00; C02F 1/42; C02F 1/32; C22B 3/42; C02F 2001/425; C02F 2101/10
USPC 210/643; 549/352; 549/214; 525/50; 521/30; 210/681; 210/670; 210/688; 210/687; 210/679

(57) ABSTRACT

A method of separating one or more valuable metal cations from an ionic solution by (a) contacting the ionic solution with an activated photoisomerizable host molecule containing a photoisomerizable moiety and a host moiety, where the photoisomerizable moiety has first and second states, and where the host moiety has a greater affinity for a metal cation when the photoisomerizable moiety is in the first state (active binding state) than when the photoisomerizable moiety is in the second state (release state), so that an ion-host molecule association is formed, and (b) separating the ion-host molecule association from the ionic solution. Also disclosed are photoisomerizable host molecules, a method of recovering valuable metals from a waste stream using the photoisomerizable host molecules, and an apparatus comprising a photoisomerizable host molecule attached to a support.

### FIELD OF THE INVENTION

The invention is related to the separation of specific metals from ionic solutions, including the recovery of metal values from industrial waste streams. The invention is also related to the use of photoisomerizable host molecules to selectively bind and release specific metal ions, particularly in the presence of other, potentially interfering metal ions.

### BACKGROUND OF THE INVENTION

Waste streams of soluble cations derived from mining or other industrial operations are typically overlooked as useful feedstocks for the extraction of precious metal cations and other types of metal cations such as rare earth and actinide species. Frequently, it is difficult to isolate a desired metal cation by precipitation from aqueous solution because the concentrations of the desired cation is on the order of a few hundred ppm while other species may be present in concentrations on the order of several percent. When the desired ion is precipitated the precipitation of all other species must be suppressed, but even when that happens the species remaining in solution can adsorb onto the surfaces of the precipitate.

Even if the desired separation is possible, large volumes of material must be subjected to unit operations such as centrifugation or filtration followed by washing the precipitate free of adsorbed species. Frequently, these processes provide low recoveries, discharging much of the valuable chemical species in the chemical waste streams. Alternative proc-esses employ liquid extraction involving nonaqueous fluids in conjunction with chelation methods. There are also non-conventional methods such as ion flotation, where a surface active reagent is added to the solution and attracts the colligend (non-surface active metal ion (or complex) of interest) to the vapor-liquid interface for removal as a foam phase.

While these methods can be effective, the toxicity of the required organic solvents poses a significant health hazard. Ion exchange resins avoid this problem since they can be fine-tuned to extract the ion of interest from solution by passing the solution over the resin bed. The ions trapped by the resin can be isolated by a second washing step where a concentrated solution of a different ion displaces the ion captured by the resin into the washing solution. However, this washing step leads to degradation and fouling of the resin, and thereby limits its lifetime.

Thus, there continues to be a need for more efficient methods of recovering valuable metals from ionic solutions, in particular from waste streams derived from mining or other industrial operations.

### BRIEF SUMMARY OF THE INVENTION

One aspect of the invention is directed to a method of separating one or more metal cations from an ionic solution, the method comprising the steps of:
(a) contacting the ionic solution with a photoisomerizable host molecule comprising a photoisomerizable moiety and a host moiety, where the photoisomerizable moiety has first and second states, and wherein the host moiety has a greater affinity for a metal cation when the photoisomerizable moiety is in the first state (active binding state) than when the photoisomerizable moiety is in the second state (release state), so that an ion-host molecule association is formed; and
(b) separating the ion-host molecule association from said ionic solution.

Another aspect of the present invention is directed to a method of separating one or more metal cations from an ionic solution, the method comprising the steps of:
(a) contacting the ionic solution with an activated photoisomerizable host molecule so that an ion-host molecule association is formed, wherein the host molecule has a structure selected from the group consisting of Formulae (Ia) to (Id):

A¹-X¹-A² (Ia)

A¹-(X¹-)ₙA² (Ib)

A¹-((X¹-)ₙA²)_{n′} (Ic)

(A¹-X¹)ₘ-A²-((X²-)ₙA³)_{n′}-(X³-A⁴)_{m′} (Id)
where n and n′ are independently selected from an integer between 1 and 100, inclusive; m and m′ are independently selected from an integer between 0 and 10,000,000, inclusive; A¹, A², A³ and A⁴ are independently selected from the group consisting of host moieties that selectively bind or bond said one or more metal cations; and X¹, X², and X³ are independently selected from the group consisting of groups that photoisomerize to or from an active binding state configuration in which at least one of the active binding state host moieties selectively binds or bonds the one or more metal cations of interest; and
(b) separating the ion-host molecule association from the ionic solution;
   where, when the photoisomerizable host molecule is in its active binding state configuration, the host moieties selectively bind one or more metal cations selected from the group consisting of Group II metals, Group III metals, rare earth metals, transition metals, coinage metals, platinum group metals (Os, Ir, Ru, Rh, Pt, Pd), metalloids (B, Si, As, Te and As), main group 13 metals, main group 14 metals, main group 15 metals, main group 16 metals and actinides.

In one embodiment, the main group 13 metals are Al, Ga, In and Ti. In another embodi-ment, the main group 14 metal is Pb. In yet another embodiment, the main group 15 metal is Bi. In another embodiment, the main group 16 metal is Po.

The method can further comprise the step of:
(c) recovering the bound metal cation from said ion-host molecule association.

The method can still further comprise the step of:
(d) recovering the photoisomerizable host molecule.

A¹, A², A³ and A⁴ are preferably cation-binding or bonding moieties independently selected from macrocyclic molecules, chelating agents, complexing agents and metal organic frameworks that selectively bind or bond said cations to be separated from the ionic solution. Macrocyclic molecules can be independently selected from, without limitation, the group consisting of crown ethers, cryptates, cryptands, and cyclodextrins. Chelating agents can be independently selected from, without limitation, carboxylates (e.g., acetate, stearate, acrylates, polycarboxylates, etc.), aminopolycarboxylates (e.g., EDTA, DOTA, etc.), polyalkene amines (e.g., ethylene diamine, DETA, TETA, TEPA, PEHA, etc.), acetoacetonates, diols (e.g., catecholates, ethylene glycol, etc.), phosphon-ates (e.g., DMMP, NTMP, HEDP, etc.), polyols, polyesters, and naturally occurring chelating agents that can be isolated from yeast, grass, legumes, or other natural sources (e.g., phytochelatins (PC2-PC11).

The photoisomerizable groups, X¹, X² and X³ of Formula (Ia) to (Id) are independently selected from the group consisting of Formula (II):

R¹—R²—B¹═B²—R³—R⁴ (II)

where B¹ and B² are independently selected from CR or N, where R is H, lower alkyl, lower haloalkyl, halogen, lower alkoxy or lower haloalkoxy;
R¹ and R⁴ are independently selected from aryl or heteroaryl; and
R² and R³ are independently selected from a bond, O, S(O)_{n″}, where n″=0-2, NR, (CH₂)_{m″}, where m″=1-12, or (CH(R″)CH₂O)_{m″}, where R″ is H or lower alkyl.

In preferred embodiments of the invention, the photoisomerizable X groups are selected from —N═N—, —CH—CH—, —N═CH— and —CH═N—.

Preferably the photoisomerizable X groups have the structure of Formula (II), where R¹ and R⁴ are phenyl, R² and R³ are each a bond, and B¹ and B² are nitrogen (—N═N—; azobenzene); or R¹ and R⁴ are phenyl, R² and R³ are each a bond, and B¹ and B² are CH (—CH═CH—; stilbene).

The ionic solution can further comprise alkali and/or alkaline earth and/or iron cations, and the host moieties have a greater binding affinity for at least one of the other (valuable) cations in the ionic solution.

The photoisomerizable host molecule can be covalently bonded to a particle or substrate support, such as a metallic and/or a ceramic and/or a polymeric and/or an organic material. Steps (a) and (b) of the above method can be performed within a column which contains the particles or support.

Further, the photoisomerizable host molecule can be dissolved in, suspended in or supported by a medium that is immiscible with the ionic solution. The medium can be a liquid membrane or a chromatography stationary phase. The stationary phase can be an ion exchange resin.

The method of the invention selectively binds or bonds valuable metal cations, even in the presence of about 1% to about 10% by weight of other ionic species. The valuable metal cations include rare earths, transition metals, coinage metals, and platinum group metals. In one preferred embodiment the rare earth metal cation is scandium.

Another aspect of the invention is directed to a method of recovering valuable metals from a waste stream, comprising the steps of:
(a) contacting said waste stream with a photoisomerizable host molecule comprising a photoisomerizable moiety and a host moiety, wherein the photoisomerizable moiety has first and second states, and wherein the host moiety has a greater affinity for said metal ions when the photoisomerizable moiety is in the first state (active binding state) than when the photoisomerizable moiety is in the second state (release state), to form an ion-host molecule association;
(b) separating the resulting ion-host molecule association from the waste stream; and
(c) recovering the bound metal cation from the ion-host molecule association;
   wherein the valuable metals comprise one or more metals selected from the group consisting of coinage metals, platinum group metals (Os, Ir, Ru, Rh, Pt, Pd), metalloids (B, Si, As, Te and As), main group 13 metals, main group 14 metals, main group 15 metals, main group 16 metals and actinides.

In a specific embodiment, step (a) involves contacting the waste stream with a photoisomerizable host molecule of Formula (Ia) to (Id) as shown above:

In one embodiment, the main group 13 metals are Al, Ga, In and Tl. In another embodiment, the main group 14 metal is Pb. In yet another embodiment, the main group 15 metal is Bi. In another embodiment, the main group 16 metal is Po.

The waste stream can comprise the valuable metals in concentrations of about 10 ppm to about 500 ppm. Further, the waste stream can comprise iron and/or alkali metals and/or alkaline earth metals in about 1% to about 10% by weight.

Another aspect of the invention is directed to the photoisomerizable host molecules themselves, which compounds comprise a photoisomerizable moiety and a host moiety, where the photoisomerizable moiety has first and second states, and wherein the host moiety has a greater affinity for a metal cation when the photoisomerizable moiety is in the first state (active binding state) than when the photoisomerizable moiety is in the second state (release state), where the metal cation is selected from the group consisting of Group II metals. Group III metals, rare earth metals, transition metals, coinage metals, platinum group metals, metalloids, main group 13 metals, main group 14 metals, main group 15 metals, main group 16 metals and actinides. In some embodiments of the invention the photoisomerizable host molecule has a structure selected from the group consisting of Formulae (Ia) to (Id), as defined above.

Yet another aspect of the invention is direct to an apparatus comprising the photoisomerizable host molecule, as disclosed above, attached to a support.

### BRIEF DESCRIPTION OF THE DRAWINGS

FIG. 1 shows the chemical structures of representative macrocyclic hosts which are suitable as host moieties for the photoisomerizable host molecules.

FIG. 2 depicts a representative photoisomerizable host molecule in the active binding state, binding a Sm cation, and in the release state, releasing the Sm cation.

### DETAILED DESCRIPTION OF PREFERRED EMBODIMENTS

New technology is needed which provides a higher level of selectivity of separation from ionic solution of the desired metal values, such as rare earth elements, including certain transition metals which have been historically referred to as rare earths, such as scandium. For the purposes of the present invention, the elements Sc, Y and Lu are also considered rare earth elements. In order to meet this need, one embodiment of the present invention utilizes specially designed photoisomerizable host molecules, such as crown ethers, which bind rare earth cations to form rare earth cation complexes.

The binding of rare earth cations by simple crown ethers was first observed in work involving the reaction of rare earth chlorides with 18-crown-6. Rare earth cation complexation with appropriate crown ethers has been characterized by large stability constants, indicating a high level of thermodynamic stability. No heat of reaction has been observed with the post-Gd³⁺ lanthanide cations (Tb³⁺, Dy³⁺, Ho³⁺, Er³⁺, Tm³⁺, Yb³⁺, Lu³⁺). For the earlier elements in the rare earth series (La³⁺, Ce³⁺, Pr³⁺, Nd³⁺, Sm³⁺, Eu³⁺, and Gd³⁺), all reaction enthalpies are reported to be positive. This implies that the observed stabilities are entropic in origin. With increasing atomic number, the rare earth complex stabilities reportedly decrease.

The interactions of crown ethers with rare earths are also very sensitive to the structure of the crown ether. For example, it has been found that 18-crown-6 actually binds with rare earth cations only up to Gd. Experiments with di-benzo-18-crown-6 indicated that com-plexes precipitated only with La³⁺, Ce³⁺, Pr³⁺, and Nd³⁺, and no other rare earth species. Thus different types of crown ethers having what would seem to be small structural modifications, such as the presence of benzene rings (benzo ring fusion), can alter the spectrum of lanthanide cations that effectively binds with each crown ether. Subsequent-ly, simple solvent extraction, chromatography, ion flotation, or passing through a liquid membrane can be used in conjunction with complex formation to effectively separate different sets of elements in the lanthanide series. These rare earth-selective methods are also effective in excluding other types of metal ions (interfering ions) such as transition metal cations and Group IIIa, IVa, Va, VIa, and VIIa cations. Further, alkali and alkaline earth ions can also be excluded with the proper choice of host molecule.

We have now discovered photoisomerizable host molecules represented by Formulae (Ia), (Ib), (Ic) and (Id) which selectively bind and release rare earth or other valuable cations:

A¹-X¹-A² (Ia)

A¹-(X¹-)ₙA² (Ib)

A¹-((X¹-)ₙA²)_{n′} (Ic)

(A¹-X¹)ₘ-A²-((X²-)ₙA³)_{n′}-(X³-A⁴)_{m′} (Id)

where A¹, A², A³ and A⁴ are independently selected from moieties that selectively bind or bond one or more metal cations, preferably valuable metal cations, and X¹, X², and X³ are independently selected from the group consisting photoisomerizable moieties. According to an embodiment, photoisomerizable moieties include π-electron delocalized chromo-phore photoisomerizable groups, also known in the art as photoswitches.

Further, photoisomerizable host molecules of the present invention can be designed and their selectivity can be tuned to bind any specific metal cation or group of metal cations in the periodic table.

### Valuable Metals

For the purposes of the present invention, “valuable metals” and “valuable metal cations” include Group II metals. Group III metals, rare earth metals, transition metals, coinage metals and platinum group metals (Os, Ir, Ru, Rh, Pt, Pd), metalloids (B, Si, As, Te and As), main group 13 metals, main group 14 metals, main group 15 metals, main group 16 metals and actinides.

In one embodiment, the main group 13 metals are Al, Ga, In and Tl. In another embodiment, the main group 14 metal is Pb. In yet another embodiment, the main group 15 metal is Bi. In another embodiment, the main group 16 metal is Po.

Coinage metals include gold, silver, copper and nickel. Platinum group metals include iridium, osmium, palladium, platinum, rhodium, and ruthenium. Actinides can be recovered from nuclear waste streams.

### Host Molecules

Host-guest chemistry describes complexes that are composed of two or more molecules or ions that are held together in unique structural relationships by forces other than those of full covalent bonds. Host-guest chemistry encompasses the concept of molecular recognition and interactions through non-covalent bonding. Non-covalent bonding is critical in maintaining the three-dimensional structure of large biomolecules, such as proteins and nucleic acids, and is involved in many biological processes in which large molecules bind specifically but transiently with one another. Commonly identified types of non-covalent interactions operative in host-guest chemistry include hydrogen bonding, ionic bonding, van der Waals forces and hydrophobic interactions. For the purposes of the present invention, host molecules or host moieties are defined as those structures which reversibly bind or bond a specific metal cation or group of cations by means of the host-guest interactions described above, and “bond” refers to bonding by other than covalent sharing of electrons.

Commons host moieties include, without limitation, cyclodextrins, calixarenes, cucurbiturils, porphyrins, metallacrowns, crown ethers, cryptands, zeolites, cyclotriveratrylenes, cryptophanes and carcerands. For the purposes of the present invention, chelating agents can also be considered to be host moieties. Chelating agents can be independently selected from, without limitation, carboxylates (e.g., acetate, stearate, acrylates, polycarboxylates), aminopolycarboxylates (e.g., EDTA, DOTA, etc.), polyalkene amines (e.g., ethylene diamine, DETA, TETA, TEPA, PEHA, etc.), acetoacetonates, diols (e.g, catecholates, ethylene glycol), phosphonates (e.g., DMMP, NTMP, HEDP, etc.), polyols, polyesters, and naturally occurring chelating agents that can be isolated from yeast, grass, legumes, or other natural sources (e.g., phytochelatins (PC2-PC11), etc.).

There are crown ethers reportedly selective for groups of specific cations, including Gd and Yb; Sm, Eu, Tb, and Dy; Eu, Tb, Gd; La and Eu; Er and Eu; Ce and Nd; Gd, Tb, Dy, Ho, Er, and Tm; and La, Ce, Pr, Sm, Eu, Gd, and Tb. Some compounds claim to be selective for the entire rare earth series, and others are suitable for all but one rare earth cation, such as Pm. Sc- and La-selective compounds are known. Thus, three novel solid cryptates of a cage-type N₂O₆-heteroatom hexabenzocryptand (L) with RE(III) (RE=Sc, La) chlorides and La nitrate respectively, have been prepared and characterized by IR and ¹H NMR spectroscopy, TG-DTA analyses and molar conductances. The compositions of these cryptates was determined to be RE₂Cl₆L.4H₂O (RE=Sc, La) and La₂(NO₃)₆L.2H₂O. An attractive feature of the reported rare earth cryptate molecules is their water solubility. Certain cryptate molecules can also differentiate between rare earth cations. In addition, cryptates can even differentiate between +2 and +3 oxidation states for Eu and Sm. In typical solution processes, rare earth metal separations from other cations have been considered nearly impossible to achieve. However, we have now discovered host molecules with unprecedented rare earth selectivity, such that separations of rare earths from other metals are even easier to achieve than separating rare earths from one another.

One embodiment of the present invention is directed to the use of cryptands, optionally in combination with crown ethers, as the binding or bonding moieties of photoisomerizable host molecules for selective extractions of valuable cations, due to the enhanced selectivity of cryptands. Cryptands are crown ethers that have some or all of their oxygen atoms replaced with nitrogen atoms. As for crown ethers, cryptands can be modified by the addition of photoresponsive groups, such as azo-benzene, which serve as a photo-switch. For the purposes of the present invention, crown ethers and cryptands will be referred to as “macrocyclic compounds”, or “macrocyclic hosts”, or “macrocyclics”. The group of suitable macrocyclic compounds also includes other host moieties, as disclosed for A¹, A², A³ and A⁴ of Formula (Ia) to (Id).

Although not wishing to be bound by any particular theory, in general the cation binding selectivity of the photoisomerizable host molecules of the invention is believed to depend at least on the following:

(i) the shape of the host moieties and preorganization within the host moieties,
(ii) the size-match of the host cavity to the guest cation,
(iii) the cation charge and type, and
(iv) the donor atom charge and type.

Donor atoms are generally recognized to be the heteroatoms oxygen, nitrogen and sulfur. Thus, crown ethers, containing only oxygen atoms, and their corresponding isosteres having one or more oxygen atoms replaced with a nitrogen or sulfur atom, are considered to be appropriate host moieties for the purposes of the present invention.

Although not wishing to be bound by any particular theory, it is believed that the Hard-Soft Acid-Base (HSAB) principle applies to the binding of the photoisomerizable host molecules of the invention with particular cationic species, as reflected in (iii) and (iv) above. The HSAB principle comprises at least the following elements:
1. Hard acids prefer hard bases and soft acids prefer soft bases;
2. A hard acid is a small, highly charged and non-polarizable acceptor atom;
3. A soft acid is a large, not highly charged polarizable atom;
4. A hard base is a small highly electronegative nonpolarizable donor atom;
5. A soft base is a large, highly polarizable donor atom.

Because of the relatively rigid structures of cryptands, crown ethers and related host moieties, thermodynamic stabilities of the cryptate complexes strongly depend on the match of the cation size and crown ether or cryptand cavity diameters. For example, when we go from 14-crown-4 to 21-crown-7, the cavity size changes from 1.2 to 4.3 Å in terms of the ionic radius. For cryptands, going from [1.1.1]cryptand to [3.3.3]cryptand, the cavity size changes from 1.0 to 4.8 Å. Geometry and symmetry of the binding sites are also important factors influencing the properties of cryptand complexation. One way to improve metal ion selectivity by oxygen-nitrogen donor cryptands is to provide a host that has a cavity that is size-matched with the cation, while maintaining a symmetric spherical-coordination array. High selectivity for a small cation can be obtained when the cryptand is able to form a number of six-membered chelate rings with the metal ion, while the requirements of the high-symmetric donor atom array and size-matched cavity are met. On the other hand, introduction of benzene rings and other similar groups to cryptands usually decreases metal ion binding and selectivity.

For the purposes of the present invention, cryptands can also include what are commonly known as macrobicyclic compounds and polycyclic compounds.

The general types of macrocyclic hosts can be described as:
1. Crown ethers, and their aza and thia analogs,
2. Coronands,
3. Crytands,
4. Podands,
5. Lariats (using C- or N-pivot atoms).

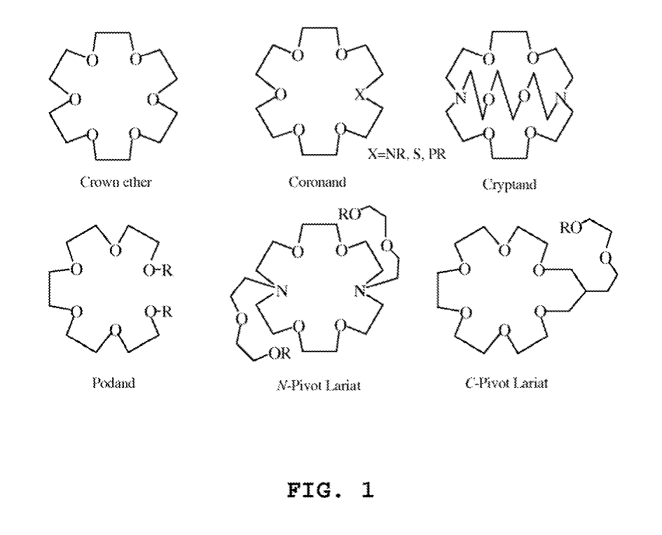

See FIG. 1 for representative examples of the above. All of these macrocyclic hosts are suitable as host moieties for the photoisomerizable host molecules of the present invention.

At best, the known molecular design principles for macrocyclic hosts are highly generalized, and do not delineate a precise approach to making rare earth-selective molecules. One challenge is to exclude certain ions known to prefer macrocyclic hosts, such as alkali and alkaline earth cations, while being selective for rare earth cations. Thus, bases such as CaCO₃ and KOH, used to neutralize many types of industrial and mining wastes, will interfere with rare earth recovery from virtually every mineral waste stream as well as many industrial waste streams, where ppm levels of rare earth and other valuable metals are ubiquitous. We have now solved this problem with the photoisomerizable host molecules disclosed herein.

From a commercial perspective, macrocyclic hosts have utility in value-added applications that permit the use of high cost tagging moieties, such as fluorescent markers for biomedical and photonic applications. However, for rare earth or other valuable metal extractions from bulk mineral and/or industrial waste streams, a suitable method to recycle the macrocyclic host is required due to high volume requirements for such bulk separations, as well as due to the cost of the macrocyclic hosts themselves.

Many separation processes that could use macrocyclic hosts do not offer the option to recycle these hosts. Isolation of rare earth complexes can be accomplished in at least two ways. First, a complex can be formed in aqueous media and then extracted with an immiscible nonaqueous solvent. However, this approach requires expensive and/or toxic nonaqueous solvents. Further, a method for destabilization of the rare earth complex and recovery of the rare earth metal is necessary. Second, the extractant, comprising the non-aqueous solvent, can be used as a crystallization medium by drying, or by the addition of another solvent to induce crystallization. Third, the dried nonaqueous extract can be freed of solvent by igniting in a furnace to burn off the solvent and form a product oxide. In all of these cases, the macrocyclic host is either destroyed or cannot be recycled, thereby leading to high process costs. Such approaches would not be suitable for a low cost large-scale process for the recovery of kilo- to mega-ton quantities of rare earth or other valuable metals. There is a need for rare earth cation extraction processes that overcome the high cost of macrocyclic hosts via either recycling-based or multi-use-based approaches.

In order to address this need, one embodiment of the present invention is directed to chemically grafting the crown ether to a high surface area porous polymer resin. Thus, the porous resin becomes capable of selectively binding the rare earth or other valuable metal species. However, a method for recovery of the bound cations is also required. Elutriation requires a long resin lifetime in order to justify its cost. The use of temperature modulation is also possible to cause the release of the bound rare earth cations as long as it does not compromise the integrity of the resin and thereby reduce its long term use. A temperature is chosen so thermal degradation of either the polymer or the photoisomerizable host molecule does not limit the useful lifetime of the resin.

### Photoresponsive Host Molecules and Photoswitches

Whether solutions or resins are utilized in an extraction, ion flotation or other partitioning process, we have now discovered a way to destabilize an ion-host molecule association so as to release the bound cation in a manner that does not degrade the host molecule. Thus, we have designed and prepared photoresponsive host molecules of Formulae (Ia) to (Id) which are selective for rare earth cations or other valuable metal cations. In some embodiments the photoisomerizable moiety contains at least one double-bonded functional group which photoisomerizes between corresponding cis- and trans-isomers, one isomer of which is characteristic of the active binding state of the photoisomerizable host molecule, and the other of which is characteristic of the release state of the photoisomerizable host molecule.

In one embodiment an azobenzene photoswitch (X¹ in Formula (Ia))

A¹-X¹-A² (Ia)

is used to link two cation-binding or bonding host moieties (A¹ and A²); the azobenzene unit photoisomerizes thereby interconverting trans- and cis-isomers. Although not wishing to be bound by any theory, it is believed that the selective photoresponsive binding behavior is attributed to conformational distortion of the host moieties, which is induced by the cis/trans-photoisomerization of the photoswitch moiety.

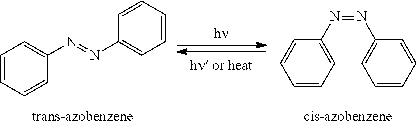 [SMILES: c1ccc(/N=N/c2ccccc2)cc1 ; c1ccc(/N=N\c2ccccc2)cc1]

Thus, an important aspect of reversible photo-controlled hosting of ions is covalent bonding of the receptor host moieties, such as a macrocyclic compound or chelating agent, to a photoswitch that is able to undergo substantial changes upon exposure to light such that the receptor host moieties are able to accommodate metal cations far more selectively than when a compound comprising only one or more host moieties, absent the photoswitch, is used. Thus, the photoswitch, such as azobenzene, distorts the host moieties to make them more selective.

Such photoresponsive host molecules can be integrated into a polymer support, such as cross-linked polystyrene beads. The polymer support serves as a fixed point to impose the conformational changes of the immobilized functional molecules. Photoresponsive complexation occurs reversibly.

The present invention is not limited to azobenzene photoswitch moieties, but can utilize any functional group which undergoes a photo-induced structural change that impacts the conformation, and hence, the cation selectivity of the host moiety when exposed to light. General types of photoswitch moieties include the following:
1. Groups that photoisomerize geometrically, such as groups containing a double-bonded functional group, exemplified by Formula (II), vide infra, including azobenzene, stilbene, and 2,2′-azopyridine;
2. Groups that photodimerize, such as polyether-containing anthracenes;
3. Groups that photoisomerize in other ways, such as spiro compounds and chromenes;
4. Groups that photocyclize, such as diarylethylenes; and
5. Groups that photodissociate.

Another suitable photoswitch moiety is a stilbene group. Heterocyclic analogs of stilbene and azobenzene are also suitable, such as 2,2′-azopyridine, as are isosteres of the central azo or ethylene double bond. In some embodiments, appropriate light-sensitive photo-switch moieties have the π-electron delocalized chromophore structure of Formula (II):

R¹—R²—B¹═B²—R³—R⁴ (II)

wherein B¹ and B² are independently selected from CR or N, where R is H, lower alkyl, lower haloalkyl, halogen, lower alkoxy or lower haloalkoxy;
R¹ and R⁴ are independently selected from aryl or heteroaryl; and
R² and R³ are independently selected from a bond, O, S(O)_{n″}, where n″=0-2, NR, (CH₂)_{m″}, where m″=1-12, or (CH(R″)CH₂O)_{m″}, where R″ is H or lower alkyl. For the azobenzene photoswitch. R¹ and R⁴ are phenyl, R² and R³ are each a bond, and B¹ and B² are N. For the stilbene photoswitch, R¹ and R⁴ are phenyl. R² and R³ are each a bond, and B¹ and B² are CH.

For the purposes of the present invention, the term “lower alkyl” denotes branched or unbranched alkyl groups of 1 to about 6 carbon atoms, preferably 1 to 4 carbon atoms. Analogous definitions apply to the terms “lower haloalkyl”, “lower alkoxy”, “lower haloalkoxy”, “lower alkylamino” and “lower dialkylamino”. The term “aryl” denotes aromatic groups of 6 to about 14 carbon atoms, optionally substituted with 1 to about 4 groups selected from lower alkyl, lower haloalkyl, halogen, lower alkoxy, lower haloalkoxy, hydroxy, nitro, amino, lower alkylamino, lower dialkylamino, B(OH)₂, and P(═O)(OH)₂. The term “heteroaryl” denotes an aromatic group of 4 to about 14 carbon atoms containing at least one heteroatom selected from the group consisting of O, S and N, optionally substituted with 1 to about 4 groups selected from lower alkyl, lower haloalkyl, halogen, lower alkoxy, lower haloalkoxy, hydroxy, nitro, amino, lower alkylamino, lower dialkylamino, B(OH)₂, and P(═O)(OH)₂.

One of the important properties of azobenzene (and derivatives and analogs thereof) is the photo-interconversion of trans- and cis-isomers, also known as photoisomerization. The two isomers can be switched with particular wavelengths of light: ultraviolet light, which corresponds to the energy gap of the π-π* (S2 state) transition, for trans-to-cis conversion, and blue light, which is equivalent to that of the n-π* (S1 state) transition, for cis-to-trans isomerization.

For a variety of reasons, the cis isomer is less stable than the trans-isomer (for instance, it has a distorted configuration and is less delocalized than the trans configuration). Thus, cis-azobenzene will thermally relax back to the trans via cis-to-trans isomerization. The trans-isomer is more stable by approximately 50 kJ/mol, and the barrier to photoisomerization is approximately 200 kJ/mol. Thus, cis-trans isomerization of the azobenzene moiety represents a model photochemical process in which one stereoisomer is favored thermally and the other stereoisomer is favored photochemically.

Visible light can better assist with the return to the trans state. However, if necessary, thermal energy can be used instead of photons; but the major disadvantage of thermal interconversion is the substantially longer switching times, which can be on the order of seconds, minutes, hours or days for thermal isomerization, versus picoseconds for optical isomerization. It is important to also note that mechanical stress and even electrostatic stimulation can also cause photoisomerization. The desired mode of isomerization is the one that induces the least amount of damage to the host molecule with prolonged use, while being capable of isomerization in a time-frame that allows the metal cation separation process to proceed in a manner that is technologically and economically attractive.

Photoisomerization to the active binding state may occur prior to contacting the photoisomerizable host molecule with the ionic solution, while contact is occurring, or after it has occurred. For those host molecules that photoisomerize to an active binding state con-figuration, methods according to the present invention therefore include steps in which the host molecule is illuminated with a wavelength of photons that photoisomerize it to the active binding state configuration, in which at least one of the active binding state host moieties selectively binds or bonds the one or more metal cations to be separated.

It is noted that the cis/trans-isomerization of certain photoswitch moieties can also be accomplished by adjusting the pH. Further, cis/trans-isomerization of certain photoswitch moieties can also be accomplished by redox chemistry, that is by adding an electron to, or removing an electron from, the photoisomerizable host molecule, or the photoswitch moiety itself.

Methods according to the present invention may bind or bond cations of interest to the host molecule and leave waste ions in the ionic solution. Other methods according to the present invention may bind or bond the undesired waste ions thereby enriching the ions of interest in the ionic solution relative to any remaining waste ions.

One embodiment of the present invention is directed to the separation of the rare earth cations by utilizing photoswitching host molecules such as (18-crown-6)-azobenzene-(18-crown-6), where A¹ and A² are 18-crown-6 and X¹ is azobenzene, either dissolved in a polar or non-polar solvent for liquid-liquid extraction, ion flotation, or attached to a polymer support for elutriation by chromatographic or liquid membrane techniques. Design of the appropriate photoisomerizable host molecules having the desired cation selectivity is an important aspect of the present invention. Aside from the use of photoisomeric hosts, metal ion selectivity can be imparted by selection of the specific macrocyclic hosts, secondary functionalities such as benzyl or benzo groups, the number, sites and geometry of the secondary functionalities on the macrocyclic host, as well as the type, number and relative placement of the photoswitching moieties.

The same can be said for more common chelating hosts such as those described above. While more common chelating hosts are considered to lack the metal ion selectivity of macrocyclics, the stereochemistry imparted by a photoswitch group can impart selectivity not seen with the chelating agent as an independent molecule. Thus, another embodiment of the invention is to utilize EDTA-azobenzene-EDTA, where A¹ and A² are EDTA and X¹ is azobenzene, either dissolved in a polar or non-polar solvent for liquid-liquid extraction, ion flotation, or attached to a polymer support for elutriation by chromatographic or liquid membrane techniques.

In another embodiment of the present invention the macrocyclic hosts are cyclodextrins, which can be natural, synthetic or semi-synthetic, and are known to solubilize lanthanides via complexation. Semi-synthetic cyclodextrins modified to include EDTA hosts also show specificity for lanthanides. To date, no photoswitching moieties, such as an azobenzene group, have been reported to modulate the cyclodextrin binding of lanthanide or rare earth cations.

In yet another embodiment of the invention, macromolecules such as metal organic frameworks (MOFs), synthesized from natural or synthetic intermediates, have been found to bind and release rare earth cations via photoisomerization. Rare earth-MOF complexes have been described in the art, but very little information relating to ion exchange is available, and there are no reports of azobenzene or another photoswitching moiety being incorporated into a MOF structure.

In some embodiments of the invention, the method of separation can also be considered to be a method of purification of the desired metal. In some embodiments, the inventive method of separation provides an enrichment of the desired metal cation of about 20% to about 100%. Preferably the enrichment of the desired metal cation is about 50% to about 99.999%. More preferably the enrichment of the desired metal cation is about 75% to about 99.99%. Still more preferably the enrichment of the desired metal cation is about 85% to about 99.9%. Most preferably the enrichment of the desired metal cation is about 90% to about 99%.

In other embodiments, the inventive method of separation provides an enrichment of the desired metal cation of about 10% to about 100%, preferably about 20% to about 90%, more preferably about 30% to about 80%, still more preferably about 40% to about 70%, and most preferably about 50% to about 60%.

A first embodiment of the invention is directed to a method of separating one or more metal cations from an ionic solution, the method comprising the steps of:
(a) contacting the ionic solution with a photoisomerizable host molecule comprising a photoisomerizable moiety and a host moiety, where the photoisomerizable moiety has first and second states, and wherein the host moiety has a greater affinity for a metal cation when the photoisomerizable moiety is in the first state (active binding state) than when the photoisomerizable moiety is in the second state (release state), so that an ion-host molecule association is formed; and
(b) separating the ion-host molecule association from said ionic solution.

A second embodiment of the invention is directed to a method of separating one or more metal cations from an ionic solution, the method comprising the steps of:
(a) contacting the ionic solution with an activated photoisomerizable host molecule so that an ion-host molecule association is formed, wherein the host molecule has a structure selected from the group consisting of Formulae (Ia) to (Id):

A¹-X¹-A² (Ia)

A¹-(X¹-)ₙA² (Ib)

A¹-((X¹-)ₙA²)_{n′} (Ic)

(A¹-X¹)ₘ-A²-((X²-)ₙA³)_{n′}-(X³-A⁴)_{m′} (Id)
where n and n′ are independently selected from an integer between 1 and 100, inclusive, preferably between 1 and 5, more preferably between 1 and 3, inclusive; m and m′ are independently selected from an integer between 0 and 10,000,000, inclusive; A¹, A², A³ and A⁴ are independently selected from the group consisting of host moieties that selectively bind or bond said one or more metal cations; and X¹, X², and X³ are independently groups that photoisomerize to or from said active binding state configuration in the presence or absence of light, as appropriate to said photoisomerizable group, in which at least one of the active binding state host moieties selectively binds or bonds the one or more metal cations to be separated; and
(b) separating the ion-host molecule association from the ionic solution; wherein, when the photoisomerizable host molecule is in its active binding state configuration, the host moieties selectively bind one or more metal cations selected from the group consisting of Group II metals, Group III metals, rare earth metals, transition metals, coinage metals and platinum group metals (Os, Ir, Ru, Rh, Pt, Pd), metalloids (B, Si, As, Te and As), main group 13 metals, main group 14 metals, main group 15 metals, main group 16 metals and actinides.

In one embodiment, the main group 13 metals are Al, Ga, In and Ti. In another embodiment, the main group 14 metal is Pb. In yet another embodiment, the main group 15 metal is Bi. In another embodiment, the main group 16 metal is Po.

For the purposes of the present invention, the term “ionic solution” generally refers to an aqueous solution comprising various ionic species, but the solution can also be aqueous OR organic, such as aqueous methanol or aqueous ethylene glycol, or organic. Suitable organic solvents for the ionic solution include, without limitation, lower alcohols, such as methanol and ethanol; glycols, such as ethylene glycol, propylene glycol, 1,3-propanediol and glycerol; glycol derivatives, such as 2-methoxyethanol; polyethers, such as poly-ethylene glycol and polypropylene glycol; and end-capped polyethers, such as methylated polyethylene glycols. Suitable waste streams for which the invention is useful for separa-ting valuable cations, include, without limitation, those derived from mining, nuclear, catalyzed reactions or other industrial operations.

In some embodiments, m and m′ above are integers independently selected from 0 to 1,000,000; in some embodiments m and m′ are integers independently selected from 1 to 10,000; in some embodiments m and m′ are integers independently selected from 10 to 1000; in other embodiments m and m′ are integers independently selected from 0 to 6; in other embodiments m and m′ are integers independently selected from 0 to 3; in other embodiments m and m′ are integers independently selected from 1 to 6; in other embodiments m and m′ are integers independently selected from 1 to 3.

Another embodiment of the present invention further comprises the step of:
(c) recovering the bound metal cation from the ion-host molecule association.

Yet another embodiment of the invention further comprises the step of:
(d) recovering the photoisomerizable host molecule.

In another embodiment of the invention, host moieties A¹, A², A³ and A⁴ are cation-binding host moieties independently selected from the group consisting of macrocyclic molecules, chelating agents, complexing agents and metal organic frameworks that selectively bind said cations to be separated from the ionic solution. The macrocyclic molecules can be independently selected from the group consisting of crown ethers, cryptates, cryptands, and cyclodextrins. The chelating agents can be independently selected from, without limitation, carboxylates (e.g., acetate, stearate, acrylates, poly-carboxylates, etc.), aminopolycarboxylates (e.g., EDTA. DOTA, etc.), polyalkene amines (e.g., ethylene diamine, DETA, TETA, TEPA, PEHA, etc.), acetoacetonates, diols (e.g. catecholates, ethylene glycol), phosphonates (e.g., DMMP, NTMP, HEDP, etc.), polyols, polyesters, and naturally occurring chelating agents that can be isolated from yeast, grass, legumes, or other natural sources (e.g., phytochelatins (PC2-PC11)).

In another embodiment of the invention, the photoisomerizable X groups X¹, X² and X³ are independently selected from the group consisting of Formula (II):

R¹—R²—B¹═B²—R³—R⁴ (II)

where B¹ and B² are independently selected from CR or N, where R is H, lower alkyl, lower haloalkyl, halogen, lower alkoxy or lower haloalkoxy;
R¹ and R⁴ are independently selected from aryl or heteroaryl; and
R² and R³ are independently selected from a bond, O, S(O)_{n″}, where n″=0-2, NR, (CH₂)_{m″}, where m″=1-12, or (CH(R″)CH₂O)_{m″}, where R″ is H or lower alkyl.

In one embodiment, the photoisomerizable group X is selected from —N═N—, —CH═CH—, —N═CH— and —CH═N—. Preferably X is chosen so that R¹ and R⁴ are phenyl, R² and R³ are each a bond, and B¹ and B² are nitrogen (—N═N—; azobenzene group); or R¹ and R⁴ are phenyl, R² and R³ are each a bond, and B¹ and B² are CH (CH═CH—; stilbene group).

In one embodiment of the invention the ionic solution further comprises alkali and/or alkaline earth and/or iron cations, and the host moieties have a greater binding affinity for at least one of the other cations in the ionic solution.

In another embodiment of the invention the photoisomerizable host molecule is covalently bonded to a particle or substrate support. The particle or substrate support can comprise a metallic and/or a ceramic and/or a polymeric and/or an organic material.

One embodiment of the invention is directed to a method wherein steps (a) and (b) are performed within a column containing the particles or support.

In one embodiment of the invention the photoisomerizable host molecule is dissolved in, suspended in or supported by a medium that is immiscible with the ionic solution. This medium can be a liquid membrane in certain embodiments. In other embodiments this medium is a chromatography stationary phase. In some embodiments the stationary phase is an ion exchange resin.

In some embodiments of the invention, when the photoisomerizable host molecule is in its active binding state configuration, at least one host moiety selectively binds or bonds rare earth metal cations. In one preferred embodiment of the invention the rare earth metal cation is scandium.

In other embodiments of the invention the rare earth metal cation is selected from the group consisting of lanthanum, cerium, praseodymium, neodymium, promethium, samarium, europium, gadolinium, terbium, dysprosium, holmium, erbium, thulium, ytterbium, lutetium, scandium and yttrium.

In other embodiments of the invention at least one host moiety selectively binds or bonds ppm concentrations of rare earth metal cations in the presence of about 1% to about 10% by weight of other ionic species, preferably about 1% to about 5%, most preferably about 1% to about 2% of other ionic species. In a preferred embodiment of the invention, when the photoisomerizable host molecule is in its active binding state configuration, at least one host moiety selectively binds or bonds ppm concentrations of transition metal cations in the presence of about 1% to about 10% by weight of other ionic species, preferably about 1% to about 5%, most preferably about 1% to about 2% of other ionic species.

In another preferred embodiment of the invention, when the photoisomerizable host molecule is in its active binding state configuration, at least one host moiety selectively binds or bonds ppm concentrations of actinide cations in the presence of about 1% to about 10% by weight of other ionic species, preferably about 1% to about 5%, most preferably about 1% to about 2% of other ionic species.

In still another preferred embodiment of the invention, when the photoisomerizable host molecule is in its active binding state configuration, at least one host moiety selectively binds or bonds ppm concentrations of coinage metal cations in the presence of about 1% to about 10% by weight of other ionic species, preferably about 1% to about 5%, most preferably about 1% to about 2% of other ionic species. In another preferred embodiment of the invention, when the photoisomerizable host molecule is in its active binding state configuration, at least one host moiety selectively binds or bonds ppm concentrations of platinum group metal cations in the presence of about 1% to about 10% by weight of other ionic species, preferably about 1% to about 5%, most preferably about 1% to about 2% of other ionic species.

In one embodiment of the invention at least two host moieties are selected from the group consisting of [1.1.1]cryptand and [2.1.1]cryptand. In another embodiment of the invention at least two host moieties are selected from the group consisting of [3.3.2]cryptand and [3.3.3]cryptand. In yet another embodiment of the invention at least two host moieties are selected from the group consisting of cyclen and EDTA. In still another embodiment of the invention at least two host moieties are selected from the group consisting of 15-crown-5 and [2.2.1]cryptand. In another embodiment at least two host moieties are selected from the group consisting of EDTA and DMMP. In yet another embodiment at least two host moieties are selected from the group consisting of Pinan monothia-14-crown-4 and Pinan monothia-19-crown-5.

In one embodiment of the invention at least two host moieties are crown ethers, and the photoswitch is azobenzene. In another embodiment of the invention at least two host moieties are cryptands, and the photoswitch is azobenzene. In yet another embodiment of the invention at least two host moieties are cyclodextrins, and the photoswitch is azobenzene. In still another embodiment of the invention at least two host moieties are crown ethers, and the photoswitch is stilbene. In another embodiment at least two host moieties are cryptands, and the photoswitch is stilbene. In yet another embodiment at least two host moieties are cyclodextrins, and the photoswitch is stilbene.

In a further embodiment of the invention at least one host moiety is a crown ether, another host moiety is a cryptand, and the photoswitch is azobenzene. In another embodiment of the invention at least one host moiety is a crown ether, another host moiety is a cyclodextrin, and the photoswitch is azobenzene. In yet another embodiment of the invention at least one host moiety is a cryptand, another host moiety is a cyclodextrin, and the photoswitch is azobenzene. In still another embodiment of the invention at least one host moiety is a crown ether, another host moiety is a cryptand, and the photoswitch is stilbene. In another embodiment at least one host moiety is a crown ether, another host moiety is a cyclodextrin, and the photoswitch is stilbene. In yet another embodiment at least one host moiety is a cryptand, another host moiety is a cyclodextrin, and the photoswitch is stilbene.

In certain embodiments of the invention, the A groups A¹, A², A³ and A⁴ independently comprise a macrocyclic molecule, chelating agent, complexing agent or metal organic framework that selectively binds or bonds the cations to be separated from the ionic solution.

Another aspect of the invention is directed to a method of recovering valuable metals from a waste stream, comprising the steps of:
(a) contacting said waste stream with a photoisomerizable host molecule comprising a photoisomerizable moiety and a host moiety, wherein the photoisomerizable moiety has first and second states, and wherein the host moiety has a greater affinity for said metal ions when the photoisomerizable moiety is in the first state (active binding state) than when the photoisomerizable moiety is in the second state (release state), to form an ion-host molecule association;
(b) separating the resulting ion-host molecule association from the waste stream; and
(c) recovering the bound metal cation from the ion-host molecule association:
   wherein the valuable metals comprise one or more metals selected from the group consisting of coinage metals, platinum group metals (Os, Ir, Ru, Rh, Pt, Pd), metalloids (B, Si, As, Te and As), main group 13 metals, main group 14 metals, main group 15 metals, main group 16 metals and actinides.

Yet another embodiment of the invention is directed to a method of recovering valuable metals from a waste stream, comprising the steps of:
(a) contacting the waste stream with a photoisomerizable host molecule of Formula (Ia) to (Id) to form an ion-host molecule association;
(b) separating the resulting ion-host molecule association from the waste stream; and
(c) recovering the bound metal cation from the ion-host molecule association;
   where the valuable metals comprise one or more metals selected from the group consisting of Group II metals, Group III metals, rare earth metals, transition metals, coinage metals, platinum group metals, metalloids, main group 13 metals, main group 14 metals, main group 15 metals, main group 16 metals and actinides.

In certain embodiments of the recovery methods, the photoisomerizable host molecule has a structure selected from the group consisting of Formulae (Ia) to (Id), shown above.

In a further embodiment of the invention the waste stream comprises the valuable metals in concentrations of about 10 ppm to about 500 ppm.

In yet another embodiment of the invention the waste stream comprises iron and/or alkali metals and/or alkaline earth metals in about 1% to about 10% by weight, preferably about 1% to about 5%, most preferably about 1% to about 2% by weight.

Another aspect of the invention is directed to the photoisomerizable host molecules themselves, which compounds comprise a photoisomerizable moiety and a host moiety, where the photoisomerizable moiety has first and second states, and wherein the host moiety has a greater affinity for a metal cation when the photoisomerizable moiety is in the first state (active binding state) than when the photoisomerizable moiety is in the second state (release state), where the metal cation is selected from the group consisting of Group II metals, Group III metals, rare earth metals, transition metals, coinage metals, platinum group metals, metalloids, main group 13 metals, main group 14 metals, main group 15 metals, main group 16 metals and actinides. In some embodiments of the invention the photoisomerizable host molecule has a structure selected from the group consisting of Formulae (Ia) to (Id), as defined above.

Yet another aspect of the invention is direct to an apparatus comprising the photoisomerizable host molecule, as disclosed above, attached to a support. The photoisomerizable host molecule can be covalently bonded to said support, or attached via non-covalent bonds. The support can comprise a metallic and/or a ceramic and/or a polymeric and/or an organic material. Further the support can be a chromatography stationary phase, such as an ion exchange resin.

### EXAMPLES

The following examples are intended be illustrative of the preferred embodiments of the invention, and do not limit the scope of the invention in any way.

### Example 1

### Photoisomerizable Host Molecule for Photo-Extraction of a Rare Earth Ion

### Synthesis of a bis(crown ether), benzo-15-crown-5

The bis(crown ether), benzo-15-crown-5, is prepared from 4′-nitrobenzo-15-crown-5 by zinc powder reduction in the presence of KOH. Benzo-15-crown-5 is synthesized from 4′-nitrobenzo-15-crown-5 as follows: One gram of NaOH in 1 mL of water and 5.1 g (0.33 mol) of 4′-nitrobenzo-15-crown-5 in 30 mL of benzene are heated at 70-80° C. The solution is stirred vigorously, and 16 g of KOH and ca. 4 g of zinc powder were added. After 5 h, the hot solution is filtered and the solid is washed with 30 mL of methanol. Air is introduced into the combined solution for 4 h. The solution is then acidified using concentrated hydrochloric acid, precipitated KCl being filtered off. The resultant filtrate is concentrated in vacuo. Benzo-15-crown-5 is isolated from the residual solid by chrom-atography (silica gel, 3:1 chloroform-acetic acid). This provides a compound of mp 187-188° C. (yellow needles); yield 9.1% IR (KBr disk) q.,+1590, VCOC 1120-1140 cm-¹; mass spectrum: m/z 563(M⁺). Anal. (C₂₈H₃₅N₂0₁₀): C, H, N.

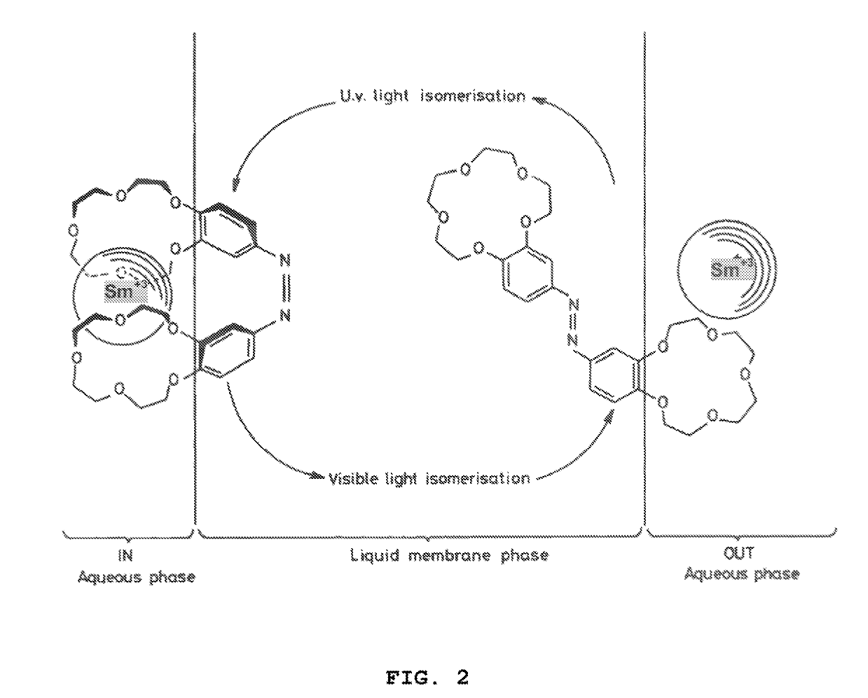

The bis(crown ether) compound is placed in a non-aqueous phase such as o-dichlorobenzene as used in a liquid membrane, as depicted in FIG. 2. The molecule is shown to capture Sm³⁺ from an aqueous phase containing 300 ppm Sm³⁺, 5 wt % Fe³⁺ and 700 ppm Al³⁺ in the “In Phase” and transfer it to the “Out Phase”. Photoisomerization is achieved with a 600 watt mercury UV lamp placed approximately 10 cm away from the reaction vessel for a 4 h period to capture the Sm³⁺. Transfer of the Sm³⁺ is accomplished by irradiation with a Xe lamp for 4 hours. The transfer and purification is determined by chemical analysis of the “In Phase” and “Out Phase” using multi-element inductively coupled plasma spectroscopy. Ion chromatography is used to check ICP results.

An analogous procedure using cryptands instead of crown ethers connected to the azobenzene structure provides compounds selective for other rare earth ions, such as Sm³⁺ or Sm²⁺.

What is claimed is:

1. A method of separating one or more metal cations from an ionic solution, the method comprising the steps of:
(a) contacting said ionic solution with a photoisomerizable host molecule comprising a photoisomerizable moiety and a host moiety, wherein the photoisomerizable moiety has first and second states, and wherein the host moiety has a greater affinity for a metal cation when the photoisomerizable moiety is in the first state (active binding state) than when the photoisomerizable moiety is in the second state (release state), so that an ion-host molecule association is formed; and
(b) separating said ion-host molecule association from said ionic solution.

2. The method of claim 1, further comprising the step of:
(c) recovering the bound metal cation from said ion-host molecule association.

3. The method of claim 2, further comprising the step of:
(d) recovering the photoisomerizable host molecule.

4. The method of claim 1, wherein said photoisomerizable host molecule has a structure selected from the group consisting of Formulae (Ia) to (Id):
A¹-X¹-A² (Ia)
A¹-(X¹-)ₙA² (Ib)
A¹-((X¹-)ₙA²)_{n′} (Ic)
(A¹-X¹)ₘ-A²-((X²-)ₘA³)_{n′}-(X³-A⁴)_{m′} (Id)
wherein n and n′ are independently selected from an integer between 1 and 100, inclusive; m and m′ are independently selected from an integer between 0 and 100,000,000, inclusive; A¹, A², A³ and A⁴ are independently selected from the group consisting of host moieties that selectively bind or bond said one or more metal cations to be separated; and X¹, X², and X³ are independently selected from the group consisting of groups that photoisomerize to or from an active binding state configuration in the presence or absence of light, as appropriate to said photoisomerizable group, in which at least one of said host moieties selectively binds or bonds said one or more metal cations.

5. The method of claim 4, wherein, when said photoisomerizable host molecule is in an active binding state configuration, said host moieties selectively bind or bond one or more metal cations selected from the group consisting of Group II metals, Group III metals, rare earth metals, transition metals, coinage metals, platinum group metals, metalloids, main group 13 metals, main group 14 metals, main group 15 metals, main group 16 metals and actinides.

6. The method of claim 4, wherein A¹, A², A³ and A⁴ are cation-binding moieties independently selected from the group consisting of macrocyclic molecules, chelating agents, complexing agents and metal organic frameworks that selectively bind said cations to be separated from said solution.

7. The method of claim 6, wherein A¹, A², A³ and A⁴ are macrocyclic molecules independently selected from the group consisting of crown ethers, cryptates, cryptands, and cyclodextrins.

8. The method of claim 6, wherein A¹, A², A³ and A⁴ are chelating agents independently selected from the group consisting of carboxylates, aminopolycarboxylates, polyalkene amines, acetoacetonates, diols, phosphonates, polyols, polyesters, and naturally occurring chelating agents.

9. The method of claim 4, wherein X¹, X² and X³ are independently selected from the group consisting of Formula (II):
R¹—R²—B¹═B²—R³—R⁴ (II)
wherein B¹ and B² are independently selected from CR or N, where R is H, lower alkyl, lower haloalkyl, halogen, lower alkoxy or lower haloalkoxy;
R¹ and R⁴ are independently selected from aryl or heteroaryl; and
R² and R³ are independently selected from a bond, O, S(O)_{n″}, where n″=0-2, NR, (CH₂)_{m″}, where m″=1-12, or (CH(R″)CH₂O)_{m″}, where R″ is H or lower alkyl.

10. The method of claim 9, wherein X is —N═N—, —CH═CH—, —N═CH— or —CH═N—.

11. The method of claim 1, wherein said ionic solution further comprises alkali and/or alkaline earth and/or iron cations, and said host moieties have a greater binding affinity for at least one of the other cations in said solution.

12. The method of claim 1, wherein said photoisomerizable host molecule is covalently bonded to particles or a substrate support.

13. The method of claim 12, wherein said particles or substrate support comprises a metallic and/or a ceramic and/or a polymeric and/or an organic material.

14. The method of claim 12, wherein steps (a) and (b) are performed within a column containing said particles or support.

15. The method of claim 1, wherein said photoisomerizable host molecule is dissolved in, suspended in or supported by a medium that is immiscible with said ionic solution.

16. The method of claim 15, wherein said medium is a liquid membrane.

17. The method of claim 15, wherein said medium is a chromatography stationary phase.

18. The method of claim 17, wherein said stationary phase is an ion exchange resin.

19. The method of claim 1, wherein when said photoisomerizable host molecule is in said active binding state configuration, at least one host moiety selectively binds or bonds rare earth metal cations.

20. The method of claim 19, wherein at least one host moiety selectively binds or bonds ppm concentrations of rare earth metal cations in the presence of about 1% to about 10% by weight of other ionic species.

21. The method of claim 20, wherein said rare earth metal cation is scandium.

22. The method of claim 1, wherein when said photoisomerizable host molecule is in said active binding state configuration, at least one host moiety selectively binds or bonds ppm concentrations of transition metal cations in the presence of about 1% to about 10% by weight of other ionic species.

23. The method of claim 1, wherein when said photoisomerizable host molecule is in said active binding state configuration, at least one host moiety selectively binds or bonds ppm concentrations of actinide cations in the presence of about 1% to about 10% by weight of other ionic species.

24. The method of claim 1, wherein when said photoisomerizable host molecule is in said active binding state configuration, at least one host moiety selectively binds or bonds ppm concentrations of coinage metal cations in the presence of about 1% to about 10% by weight of other ionic species.

25. The method of claim 1, wherein when said photoisomerizable host molecule is in said active binding state configuration, at least one host moiety selectively binds or bonds ppm concentrations of platinum group metal cations in the presence of about 1% to about 10% by weight of other ionic species.

26. The method of claim 9, wherein R¹ and R⁴ are phenyl, R² and R³ are each a bond, and B¹ and B² are nitrogen.

27. The method of claim 9, wherein R¹ and R⁴ are phenyl, R² and R³ are each a bond, and B¹ and B² are CH.

28. The method of claim 4, wherein at least two host moieties are selected from the group consisting of [1.1.1]cryptand and [2.1.1]cryptand.

29. The method of claim 28, wherein at least two host moieties are selected from the group consisting of [3.3.2]cryptand and [3.3.3]cryptand.

30. The method of claim 28, wherein at least two host moieties are selected from the group consisting of cyclen and EDTA.

31. The method of claim 28, wherein at least two host moieties are selected from the group consisting of 15-crown-5 and [2.1.1]cryptand.

32. The method of claim 30, wherein at least two host moieties are selected from the group consisting of EDTA and DMMP.

33. The method of claim 31, wherein at least two host moieties are Pinan monothia-14-crown-4 and Pinan monothia-19-crown-5.

34. The method of claim 19, wherein said rare earth cation is selected from the group consisting of lanthanum, cerium, praseodymium, neodymium, promethium, samarium, europium, gadolinium, terbium, dysprosium, holmium, erbium, thulium, ytterbium, lutetium, scandium and yttrium.

35. The method of claim 4, wherein A¹, A², A³ and A⁴ independently comprise a macrocyclic molecule, chelating agent, complexing agent or metal organic framework that selectively binds or bonds said cations to be separated from said solution.

36. A method of recovering valuable metal ions from a waste stream, comprising the steps of:
(a) contacting said waste stream with a photoisomerizable host molecule comprising a photoisomerizable moiety and a host moiety, wherein the photoisomerizable moiety has first and second states, and wherein the host moiety has a greater affinity for said metal ions when the photoisomerizable moiety is in the first state (active binding state) than when the photoisomerizable moiety is in the second state (release state), to form an ion-host molecule association;
(b) separating said ion-host molecule association from said waste stream; and
(c) recovering the bound metal ions from said ion-host molecule association;
wherein said valuable metals comprise one or more metals selected from the group consisting of Group II metals, Group III metals, rare earth metals, transition metals, coinage metals, platinum group metals, metalloids, main group 13 metals, main group 14 metals, main group 15 metals, main group 16 metals and actinides.

37. The method of claim 36, wherein said waste stream comprises said valuable metals in concentrations of about 10 ppm to about 500 ppm.

38. The method of claim 36, wherein said waste stream comprises iron and/or alkali metals and/or alkaline earth metals in about 1% to about 10% by weight.

39. The method of claim 36, wherein said photoisomerizable host molecule has a structure selected from the group consisting of Formulae (Ia) to (Id):
A¹-X¹-A² (Ia)
A¹-(X¹-)ₙA² (Ib)
A¹-((X¹-)ₙA²)_{n′} (Ic)
(A¹-X¹)ₘ-A²-((X²-)ₙA³)_{n′-(X}³-A⁴)_{m′} (Id)
wherein n and n′ are independently selected from an integer between 1 and 100, inclusive; m and m′ are independently selected from an integer between 0 and 100,000,000, inclusive; A¹, A², A³ and A⁴ are independently selected from the group consisting of host moieties that selectively bind or bond said one or more metal cations to be separated; and X¹, X², and X³ are independently selected from the group consisting of groups that photoisomerize to or from an active binding state configuration in the presence or absence of light, as appropriate to said photoisomerizable group, in which at least one of said host moieties selectively binds or bonds said one or more metal cations.

40. A compound comprising a photoisomerizable host molecule, comprising a photoisomerizable moiety and a host moiety, wherein the photoisomerizable moiety has first and second states, and wherein the host moiety has a greater affinity for a metal cation when the photoisomerizable moiety is in the first state (active binding state) than when the photoisomerizable moiety is in the second state (release state), wherein said metal cation is selected from the group consisting of Group II metals, Group III metals, rare earth metals, transition metals, coinage metals, platinum group metals, metalloids, main group 13 metals, main group 14 metals, main group 15 metals, main group 16 metals and actinides.

41. The compound of claim 40, where the compound has a structure selected from the group consisting of Formulae (Ia) to (Id):
A¹-X¹-A² (Ia)
A¹-(X¹-)ₙA² (Ib)
A¹-((X¹-)ₙA²)_{n′} (Ic)
(A¹-X¹)ₘA²-((X²-)ₙA³)_{n′}-(X³-A⁴)_{m′} (Id)
wherein n and n′ are independently selected from an integer between 1 and 100, inclusive; m and m′ are independently selected from an integer between 0 and 100,000,000, inclusive; A¹, A², A³ and A⁴ are independently selected from the group consisting of host moieties that selectively bind or bond one or more metal cations to be separated; and X¹, X², and X³ are independently selected from the group consisting of groups that photoisomerize to or from an active binding state configuration in the presence or absence of light, as appropriate to said photoisomerizable group, in which at least one of said host moieties selectively binds or bonds said one or more metal cations.

42. The compound of claim 41, wherein A¹, A², A³ and A⁴ are cation-binding moieties independently selected from the group consisting of macrocyclic molecules, chelating agents, complexing agents and metal organic frameworks that selectively bind said cations to be separated from said solution.

43. The compound of claim 41, wherein A¹, A², A³ and A⁴ are macrocyclic molecules independently selected from the group consisting of crown ethers, cryptates, cryptands, and cyclodextrins.

44. The compound of claim 41, wherein A¹, A², A³ and A⁴ are chelating agents independently selected from the group consisting of carboxylates, aminopolycarboxylates, polyalkene amines, acetoacetonates, diols, phosphonates, polyols, polyesters, and naturally occurring chelating agents.

45. The compound of claim 41, wherein X¹, X² and X³ are independently selected from the group consisting of Formula (II):
R¹—R²—B¹═B²—R³—R⁴ (II)
wherein B¹ and B² are independently selected from CR or N, where R is H, lower alkyl, lower haloalkyl, halogen, lower alkoxy or lower haloalkoxy;
R¹ and R⁴ are independently selected from aryl or heteroaryl; and
R² and R³ are independently selected from a bond, O, S(O)_{n″}, where n″=0-2, NR, (CH₂)_{m″}, where m″=1-12, or (CH(R″)CH₂O)_{m″}, where R″ is H or lower alkyl.

46. The compound of claim 45, wherein X is —N═N—, —CH═CH—, —N═CH— or —CH═N—.

47. The compound of claim 45, wherein R¹ and R⁴ are phenyl, R² and R³ are each a bond, and B¹ and B² are nitrogen.

48. The compound of claim 45, wherein R¹ and R⁴ are phenyl, R² and R³ are each a bond, and B¹ and B² are CH.

49. The compound of claim 41, wherein A¹, A², A³ and A⁴ independently comprise a macrocyclic molecule, chelating agent, complexing agent or metal organic framework that selectively binds or bonds said cations to be separated from said solution.

50. An apparatus, comprising the compound of claim 40 attached to a support.

51. The apparatus of claim 50, wherein said compound comprises a photoisomerizable host molecule has a structure selected from the group consisting of Formulae (Ia) to (Id):
A¹-X¹-A² (Ia)
A¹-(X¹-)ₙA² (Ib)
A¹-((X¹-)ₙA²)_{n′} (Ic)
(A¹-X¹)ₘ-A²-((X²-)ₙA³)_{n′}-(X³-A⁴)_{m′} (Id)
wherein n and n′ are independently selected from an integer between 1 and 100, inclusive; m and m′ are independently selected from an integer between 0 and 100,000,000, inclusive; A¹, A², A³ and A⁴ are independently selected from the group consisting of host moieties that selectively bind or bond said one or more metal cations to be separated; and X¹, X², and X³ are independently selected from the group consisting of groups that photoisomerize to or from an active binding state configuration in the presence or absence of light, as appropriate to said photoisomerizable group, in which at least one of said host moieties selectively binds or bonds said one or more metal cations.

52. The apparatus of claim 51, wherein A¹, A², A³ and A⁴ are cation-binding moieties independently selected from the group consisting of macrocyclic molecules, chelating agents, complexing agents and metal organic frameworks that selectively bind said cations to be separated from said solution.

53. The apparatus of claim 51, wherein A¹, A², A³ and A⁴ are macrocyclic molecules independently selected from the group consisting of crown ethers, cryptates, cryptands, and cyclodextrins.

54. The apparatus of claim 51, wherein A¹, A², A³ and A⁴ are chelating agents independently selected from the group consisting of carboxylates, aminopolycarboxylates, polyalkene amines, acetoacetonates, diols, phosphonates, polyols, polyesters, and naturally occurring chelating agents.

55. The apparatus of claim 51, wherein X¹, X² and X³ are independently selected from the group consisting of Formula (II):
R¹—R²—B¹═B²—R³—R⁴ (II)
wherein B¹ and B² are independently selected from CR or N, where R is H, lower alkyl, lower haloalkyl, halogen, lower alkoxy or lower haloalkoxy;
R¹ and R⁴ are independently selected from aryl or heteroaryl; and
R² and R³ are independently selected from a bond, O, S(O)_{n″}, where n″=0-2, NR, (CH₂)_{m″}, where m″=1-12, or (CH(R″)CH₂O)_{m″}, where R″ is H or lower alkyl.

56. The apparatus of claim 55, wherein X is —N═N—, —CH═CH—, —N═CH— or —CH═N—.

57. The apparatus of claim 50, wherein said photoisomerizable host molecule is covalently bonded to said support.

58. The apparatus of claim 50, wherein said support comprises a metallic and/or a ceramic and/or a polymeric and/or an organic material.

59. The apparatus of claim 50, wherein said support is a chromatography stationary phase.

60. The apparatus of claim 59, wherein said stationary phase is an ion exchange resin.

61. The apparatus of claim 55, wherein R¹ and R⁴ are phenyl, R² and R³ are each a bond, and B¹ and B² are nitrogen.

62. The apparatus of claim 55, wherein R¹ and R⁴ are phenyl, R² and R³ are each a bond, and B¹ and B² are CH.

63. The apparatus of claim 51, wherein A¹, A², A³ and A⁴ independently comprise a macrocyclic molecule, chelating agent, complexing agent or metal organic framework that selectively binds or bonds said cations to be separated from said solution.
